(19) United States
(12) Patent Application Publication
(10) Pub. No.: US 2026/0258080 A1
(43) Pub. Date: Sep. 3, 2026

(54) SINGLE STEP PROCESS FOR PREPARATION OF PURE IMMUNOGLOBULIN FROM PLASMA

(71) Applicant: Aegros Ltd, New South Wales (AU)

(72) Inventors: Hari NAIR, New South Wales (AU); Stephen MAHLER, New South Wales (AU); Balarka BANERJEE, New South Wales (AU); Brian SAPUTRO, New South Wales (AU); Chrys MAOUDIS, New South Wales (AU); Christina CHEN, New South Wales (AU)

(21) Appl. No.: 19/164,844

(22) PCT Filed: Mar. 13, 2024
(86) PCT No.: PCT/AU2024/050219

§ 371 Date: Sep. 12, 2025

(30) Foreign Application Priority Data
Mar. 13, 2023 (AU) 2023900670

(51) Int. Cl. C07K 1/24; C07K 16/00

(52) U.S. Cl. CPC C07K 1/24; C07K 16/00

(57) ABSTRACT

There is provided a process for the production of at least 95% pure immunoglobulin from plasma; the process comprising applying the plasma to a first fluid stream of an electroseparation system, the electroseparation system comprising a separation membrane disposed between a cathode and an anode, the separation membrane having defined pore sizes; a first restriction membrane disposed between the cathode and the separation membrane and defining a first fluid path for a first fluid stream wherein the first restriction membrane prevents molecules in the first fluid stream contacting the cathode; a second restriction membrane disposed between the anode and the separation membrane and defining a second fluid path for a second fluid stream wherein the second restriction membrane prevents molecules in the second fluid stream contacting the anode; and applying an electric field to the fluid streams to selectively cause positively charged immunoglobulin molecules to cross the separation membrane toward the cathode and into the second fluid stream. Alternatively the process comprises applying the plasma to the second fluid stream of the electroseparation system, and applying an electric field to the fluid streams to selectively cause negatively charged molecules in the plasma to cross the separation membrane toward the anode and into the first fluid stream.

### TECHNICAL FIELD

The technology relates to separation of at least 95% pure immunoglobulin from plasma using a single electroseparation step.

### CROSS REFERENCE TO RELATED APPLICATION

This application claims priority to Australian provisional application number 2023900670 filed on 13 Mar. 2023, the contents of which are incorporated by reference in their entirety.

### BACKGROUND

Plasma comprises a number of proteins including coagulants (predominantly fibrinogen), albumin and globulin to maintain colloidal osmotic pressure at about 25 mmHg, electrolytes, immunoglobulins and various other enzymes, hormones, and vitamins.

There are numerous clinical uses of plasma or plasma components. Plasma can help fight infection and treat inflammatory and autoimmune diseases, and can promote blood clotting, prevent shock and assist with post-surgical recovery. For example, whole plasma is indicated in the treatment of hemorrhagic shock. Clotting factors can be used in the treatment of hemophilia and von Willebrand disease. Infusions of albumin can be used in the treatment of burns, liver cirrhosis, and in the management of hepatorenal syndrome. In addition, human plasma-derived alpha-1 antitrypsin has been used to treat alpha-1 antitrypsin deficiency.

Immunoglobulins derived from pooled human plasma are useful in the treatment of an expanding number of autoimmune, inflammatory, hematological and viral diseases, including COVID-19. Immunoglobulins are typically administered as intravenous immunoglobulin (IVIG).

A common method for preparing immunoglobulins from plasma uses the Cohn/Oncley fractionation procedure which separates plasma proteins by differential precipitation with cold ethanol. Plasma fractionation involves successive processing steps at defined ethanol concentrations, associated with shifts in pH, temperature, and ionic strength that result in selective precipitation of immunoglobulin G (IgG).

Further purification is achieved using at least two chromatography steps, with most schemes involving size exclusion, anion/cation exchange or affinity chromatography. Chromatography achieves greater selectivity in protein purification and recoveries are typically greater than 70% when proteins are isolated from plasma fractions. However, chromatographic approaches have the disadvantage that the resins and columns are expensive and must be sanitized between use.

The conventional plasma fractionation process has a number of disadvantages including that it is not amenable to automation, it uses ethanol which impacts on plant design, it exposes immunoglobulins to harsh conditions which negatively affect their function, solubilization of precipitated proteins is often difficult and the yield of immunoglobulins is low, usually around 50%.

The disadvantages of conventional purification schemes result in an inability to meet the high demand for plasma-derived therapeutic molecules such as IVIG. There has therefore been a concerted effort in the plasma fractionation industry to find new, improved methods for rapid fractionation of plasma and purification of IgG with high recovery and purity, whilst not compromising the biological functionality of the target protein.

### SUMMARY

In a first aspect, there is provided a process for the production of at least 95% pure immunoglobulin from plasma; the process comprising
a. applying the plasma to a first fluid stream of an electroseparation system, the electroseparation system comprising:
   a separation membrane disposed between a cathode and an anode, the separation membrane having defined pore sizes;
   a first restriction membrane disposed between the cathode and the separation membrane and defining a first fluid path for a first fluid stream wherein the first restriction membrane prevents molecules in the first fluid stream contacting the cathode;
   a second restriction membrane disposed between the anode and the separation membrane and defining a second fluid path for a second fluid stream wherein the second restriction membrane prevents molecules in the second fluid stream contacting the anode; and
   b. applying an electric field to the fluid streams to selectively cause positively charged immunoglobulin molecules to cross the separation membrane toward the cathode and into the second fluid stream; or
   a. applying the plasma to the second fluid stream of the electroseparation system, and
b. applying an electric field to the fluid streams to selectively cause negatively charged molecules in the plasma to cross the separation membrane toward the anode and into the first fluid stream.

The process of claim may further comprise the step of harvesting the at least 95% pure immunoglobulin from the first or second stream.

In one embodiment the process of claim further comprises preparing the plasma by reducing the pH of pooled plasma to about 4.5 to 7.5.

The pH can be reduced by the addition of a buffer, for example a buffer comprising MES, Bis-tris, and glycine.

The process may further comprise removing precipitate from the plasma, for example by filtration or centrifugation, preferably filtration.

In one embodiment the first fluid and/or second fluid stream comprises a buffer, for example a buffer comprising MES, Bis-tris, and glycine.

In one embodiment the pore size of the separation membrane is about from about 200 kDa to about 1500 kDa, preferably about 1000 kDa.

In one embodiment the at least 95% pure immunoglobulin is free of bacteria, virus or prions when harvested from the first or second stream.

The process may further comprise concentration of the at least 95% pure immunoglobulin.

In a second aspect there is provided a preparation comprising at least 95% pure immunoglobulin produced by the process of the first aspect. In one embodiment the immunoglobulin produced by the process has substantially the same IgG subclass distribution as the original plasma.

### Definitions

Throughout this specification, unless the context clearly requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, the term ‘consisting of’ means consisting only of.

Any discussion of documents, acts, materials, devices, articles or the like which has been included in the present specification is solely for the purpose of providing a context for the present technology. It is not to be taken as an admission that any or all of these matters form part of the prior art base or were common general knowledge in the field relevant to the present technology as it existed before the priority date of each claim of this specification.

Unless the context requires otherwise or specifically stated to the contrary, integers, steps, or elements of the technology recited herein as singular integers, steps or elements clearly encompass both singular and plural forms of the recited integers, steps or elements.

In the context of the present specification the terms ‘a’ and ‘an’ are used to refer to one or more than one (ie, at least one) of the grammatical object of the article. By way of example, reference to ‘an element’ means one element, or more than one element.

In the context of the present specification the term ‘about’ means that reference to a figure or value is not to be taken as an absolute figure or value but includes margins of variation above or below the figure or value in line with what a skilled person would understand according to the art, including within typical margins of error or instrument limitation. In other words, use of the term ‘about’ is understood to refer to a range or approximation that a person or skilled in the art would consider to be equivalent to a recited value in the context of achieving the same function or result.

Those skilled in the art will appreciate that the technology described herein is susceptible to variations and modifications other than those specifically described. It is to be understood that the technology includes all such variations and modifications. For the avoidance of doubt, the technology also includes all of the steps, features, and compounds referred to or indicated in this specification, individually or collectively, and any and all combinations of any two or more of said steps, features and compounds.

In order that the present technology may be more clearly understood, preferred embodiments will be described with reference to the following drawings and examples.

### DESCRIPTION OF EMBODIMENTS

The process described herein uses plasma, typically pooled human plasma, as a source of immunoglobulins and involves a single electro-separation step to prepare at least 95% pure immunoglobulin from pooled plasma.

### Pooled Plasma

The Plasma may be provided or prepared.

In some embodiments the preparation involves adjusting the pH of the plasma ahead of the electro-separation.

In one embodiment the pH of the plasma may be adjusted by any means known in the art such as diafiltration.

In a preferred embodiment, the plasma is prepared by diluting the pooled plasma with a buffer so that the pH of the diluted plasma is in the range of about pH 4.5 to about pH 7.5. For example, the pH of the diluted plasma is about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4 or 7.5.

It will be understood that as the pH is reduced plasma proteins with a pl around the pH of the diluted plasma will precipitate. In this regard the process may optionally comprise a precipitate removal step in which the precipitated proteins are removed from the diluted plasma for example by continual stirring followed by filtration, centrifugation or filtration, preferably filtration.

Any biological buffer that can be formulated to reduce the pH of the diluted plasma can be used in the present process. It is within the knowledge of the skilled person to select and formulate an appropriate buffer.

Examples of suitable biological buffers include MES, Bis-tris, ADA, ACES, PIPES, MOPSO, Cholamine chloride, Bis-tris propane, BES, MOPS, BES, TES, HEPES, DIPSO, MOBS, TAPSO, Acetamidoglycine, Tris, HEPPSO, POPSO, TEA, HEPPS, HEPPSO, Tricine, Glycine, Glycinamide, Glycylglycine, Bicine, HEPBES, TAPS, and combinations thereof.

In some embodiments the buffer does not contain a salt (e.g. NaCl).

The pH of the buffer may be different to the final pH of the diluted plasma.

In order to prepare the plasma the buffer is mixed with pooled plasma to form the diluted plasma.

In some embodiments the ratio of buffer: pooled plasma is from about 0.5:1 to about 4:1, for example 0:5:1, 1:1, 1.5:1, 2:1, 2.5:1, 3:1, 3.5:1 or about 4:1. Preferably the ratio is about 2.5:1, 2:1, or 2.5:1, for example about 2:1.

In one embodiment the buffer comprises a combination of MES hydrate, Bis-tris and glycine. The pH of this buffer is about 3.5-8.5. For example, the pH of the buffer can be about 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or about 8.5.

In one embodiment the buffer comprises 15.86 kg of MES hydrate, 1.66 kg of Bis-tris and 11.56 kg of glycine in total volume of 668 L. In this embodiment, the buffer is mixed with about 320 L of thawed pooled plasma to form the diluted plasma. A skilled person will be able to alter the buffer volumes to account for different amounts of pooled plasma.

Precipitates in the diluted plasma are optionally removed. Removal of the precipitated proteins may comprise allowing the precipitate to settle in a holding tank, for example a bottom drain tank. Removal of the precipitate can additionally or alternatively comprise centrifugation and/or filtration. Filtration is generally preferred as it is amenable for use with large volumes.

In some embodiments the plasma is subjected to a buffer exchange step before electroseparation. Buffer exchange can be achieved by any means known in the art, for example using filtration (e.g. diafiltration), dialysis, or chromatography. Filtration (e.g. diafiltration) is generally preferred as it is amendable for use with large volumes.

In one embodiment the buffer is exchanged for another buffer using a filtration step, for example a tangential flow filtration step. In this embodiment the buffer is exchanged for a similar of different biological buffer. The choice of buffer in this context is within the knowledge of the skilled person.

In one embodiment the MES hydrate, Bis-tris and glycine buffer used to dilute the plasma is exchanged with a MES hydrate, Bis-tris and glycine buffer of a slightly different composition. In one embodiment the exchange buffer comprises 12.29 kg of MES hydrate, 2.85 kg of Bis-tris and 16.69 kg of glycine in total volume of 1058 L. In this embodiment, the buffer is mixed with about 988 L of diluted plasma. A skilled person will be able to adjust the buffer volumes to account for different starting amounts of plasma.

In some embodiments the ratio of exchange buffer: plasma is from about 0.5:1 to about 2:1, for example 0:5:1, 0.75:1, 1:1, 1.25:1, 1.5:1, 1.75:1 or about 2:1. Preferably the ratio is about 0.75:1, 1:1, or 1.25:1, for example about 1:1.

In some embodiments is advantageous to minimize the contribution of the buffer species to the ionic strength of the buffer, this can be achieved by using a zwitterionic buffer species (e.g. a so called Goods' buffer such as MES or Tris) rather than a salt such as a sodium phosphate or ammonium chloride.

### Single Electroseparation Step

Immunoglobulins are separated from the plasma using a single electroseparation step.

Electroseparation is achieved using an electroseparation system comprising
a separation membrane disposed between a cathode and an anode, the separation membrane having defined pore sizes;
a first restriction membrane disposed between the cathode and the separation membrane and defining a first flow path for a first fluid stream wherein the first restriction membrane prevents molecules in the first fluid stream contacting the cathode;
a second restriction membrane disposed between the anode and the separation membrane and defining a second flow path for a second fluid stream wherein the second restriction membrane prevents molecules in the second fluid stream contacting the anode.

The electroseparation system may further comprise an inlet and an outlet in fluid communication with the first fluid path and an inlet and an outlet in fluid communication with the second fluid path.

In use, the first fluid stream travels along the first flow path, and the second fluid stream travels along the second flow path. An electric field is applied to the fluid streams in the flow paths to cause negatively charged molecules in the fluid streams to move towards the anode and positively charged molecules in the fluid streams to move towards the cathode. In general, molecules with size (or hydrodynamic radius) less than the pore size of the separation membrane will pass across the separation membrane from one fluid stream to the other fluid stream. The shape of a molecule also affects the passage of a charged molecule across the membrane. For example a rod shaped protein may have a hydrodynamic radius such that when moving in solution the protein is prevented from passing through the membrane.

Typically, one of the fluid streams (e.g. the first or second fluid stream) is a feed stream to which the plasma is applied and the other stream is the product stream which receives molecules that have passed through the separation membrane on the application of an electric field.

In some embodiments, the flow paths are independently a linear path, a series of linear paths linked by turns. Alternatively, the flow paths may be a zig zag, or spiral. In some embodiments, the flow paths are as long as possible to maximise the time that the fluid stream is exposed to the electric field and thereby reducing or eliminating the need to recirculate a fluid stream to achieve effective separation of immunoglobulin.

Typically, the restriction membranes are molecular barriers with a smaller defined pore size than the separation membrane. The separation membranes function to prevent proteins and other macromolecules in the fluid streams from coming in contact with the electrodes (i.e., the anode and cathode).

Suitable electroseparation systems include the Aegros HaemaFrac® system that uses the mobility of charged particles in an electric field to transport proteins across a membrane into a collection chamber or fluid stream.

In one embodiment the fluid of the first and/or second fluid stream is a buffer, for example a biological buffer as described above.

In one embodiment the buffer comprises a combination of MES hydrate, Bis-tris and glycine. For example, the buffer comprises 5.8 kg of MES hydrate, 1.3 kg of Bis-tris and 9.3 kg of glycine in total volume of 500 L. A skilled person will be able to alter the buffer volumes to account for different starting amounts of plasma.

The plasma is applied to the first fluid stream. For example the plasma can be pumped into the first fluid stream via the inlet. As the plasma enters the first fluid stream an electric field is applied to the fluid streams.

As the first fluid stream exits the first fluid path, for example via the outlet, it can be recirculated to the inlet of the first fluid path, or it may be collected.

In some embodiments the first and or second fluid stream may be cooled.

As the second fluid stream exits the second fluid path, for example via the outlet, it can be recirculated to the inlet of the second fluid path, or it may be collected.

Typical flow rates used in the process for each of the fluid streams are independently selected from around 5 to 100 ml/min. For example, the flow rate may be any one of 5, 10, 15, 20, 25, 30, 35, 40, 45, 50, 55, 60, 65, 70, 75, 80, 85, 90, 95, or 100 ml/min. In an embodiment, the flow rate is between 5 and 50 mL/min. In a further embodiment, the flow rate is about 20 mL/min.

In some embodiments, a voltage of about 50 V is applied.

In one embodiment the electric field causes positively charged immunoglobulin molecules to cross the separation membrane towards the cathode into the second fluid stream.

In an alternate embodiment the electric field causes negatively charged molecules to cross the separation membrane towards the anode into the second fluid stream.

The separation membrane comprises pores of a defined size sufficient to allow passage of immunoglobulins across the membrane. Plasma contains various immunoglobulin isotypes although Immunoglobulin G (IgG) accounts for 70% to 75% of the total immunoglobulins in plasma. IgG's have a molecular weight of approximately 150 kDa, and the pore size of the membrane is larger than this to allow the immunoglobulin to cross the membrane.

In one embodiment the pore size of the membrane is at least 200 kDa to about 1500 kDa, for example 200, 250, 300, 250, 400, 450, 500, 550, 600, 650, 700, 750, 800, 850, 900, 950, 1000, 1050, 1100, 1150, 1200, 1250, 1300, 1350, 1400, 1450, or about 1500 kDa.

In one embodiment the pore size of the membrane is at least about 800, 850, 900, 950, 1000, 1050, 1100, 1150, or 1200 kDa, preferably 900, 950, 1000, 1050, or 1100 kDa, even more preferably about 1000 kDa.

The pore size can be the average pore size, or the median pore size.

The separation membrane may be a polysulfone, polypropylene, cellulose acetate, polylactic acid, or polyacrylamide-based membrane.

In some embodiment the membrane is a hydrogel membrane, for example a polyacrylamide-based hydrogel. In other embodiments hydrogel membranes based on polyethylene glycol, or chitosan may be used.

In some embodiments the separation membrane prevents mixing of the fluid streams but allows charge molecules to pass from one fluid stream to the other.

When the electric field is applied to the fluid streams it is expected that all positively charged proteins would cross the separation membrane towards the cathode into the second fluid stream if they are smaller than the pore size of the membrane. Consequently at least 95% of the proteins that enter the second fluid stream are immunoglobulins, hence the present process is a process to produce at least 95% pure immunoglobulin from the pooled plasma.

Alternatively when the electric field is applied to the fluid streams it is expected that all negatively charged proteins would cross the separation membrane towards the anode into the second fluid stream if they are smaller than the pore size of the membrane and positively charged immunoglobulins will remain in the first fluid stream. Consequently at least 95% of the proteins that remain in the first fluid stream are immunoglobulins from the pooled plasma.

In some embodiments the immunoglobulin produced by the process is at least 95% pure, at least 96% pure, at least 97% pure, at least 98% pure or at least 99% pure. In this context % purity refers to the total amount of immunoglobulin (of all IgG subtypes) expressed as a percentage of the total amount of all proteins present in the second fluid stream.

Immunoglobulin purity can be measured by any means known in the art, for example native or denaturing polyacrylamide gel electrophoresis (e.g., SDS PAGE), analytical HPLC or mass spectrometry.

In some embodiments the yield of immunoglobulin produced by the process is at least 80% and up to 95% or more of the total immunoglobulin in the plasma or the pooled plasma. For example, the yield may be at least about 80%, for example at least 80%, 82%, 84%, 86%, 88%, 90%, 92%, 94%, 95%, 96%, 97%, 98% or at least 99%. In this context % yield refers to the amount of immunoglobulin (of all IgG subtypes) in the second fluid stream expressed as a percentage of the total amount of immunoglobulins present in the plasma or the pooled plasma.

As the pore size of the separation membrane is substantially smaller than bacteria and viruses the electroseparation step is also a viral and bacterial filtration/removal step.

The at least 95% pure immunoglobulin in the second stream can be harvested, for example by collecting the second fluid stream in a container.

It is known that processes involving ethanol fractionation can result in selective loss of some IgG subtypes, particularly IgG3. This is problematic as the IgG profile is key to ability of the plasma to neutralize viral infections such as SARS-CoV-2. For example, Kober et al. (2022) PLOS ONE 17(1): e0262162 found that hyperimmune immunoglobulins or convalescent plasma donations with high IgG3 concentrations may be a highly efficacious therapy. Viral infections in general lead to production of IgG1 and IgG3, with IgG3 appearing first in the course of the infection. IgG3 is particularly effective in the induction of effector functions.

It is also known that individuals with low IgM or IgG3 levels have an increased risk of developing post-acute coronavirus disease 2019 (COVID-19) syndrome (PACS) or long COVID (see for example Cervia et al, Nature Communications (2022) 13:446)

In this regard immunoglobulin preparations made by processes that involve ethanol precipitation are often depleted in IgG3 (see for example Table 1). Caprylate precipitation removes non-IgG leaving IgG in solution and provides an IgG subclass distribution that mirrors the distribution in the plasma. However, bioprocessing is more complex, requiring further mild purification steps to ensure preservation of antibody activity.

TABLE 1
IgG subclass profile of commercially
available immunoglobulin preparations

| | | IgG Subclass |
|---|---|---|
| Product | Manufacturing process | profile |
| Intragam | Chromatographic fractionation | IgG1 47.6-56.2% |
| 10 | | IgG2 41.5-49.5% |
| | | IgG3 1.3-1.6% |
| | | IgG4 0.9-1.3% |
| Flebogamma | Cold alcohol fractionation, polyethylene | IgG1 66.6% |
| 5% DIF | glycol precipitation, ion exchange | IgG2 28.5% |
| | chromatography and low pH treatment | IgG3 2.7% |
| | | IgG4 2.2% |
| Flebogamma | Cold alcohol fractionation, polyethylene | IgG1 66.6% |
| 10% DIF | glycol precipitation, ion exchange | IgG2 27.9% |
| | chromatography and low pH treatment | IgG3 3.0% |
| | | IgG4 2.5% |
| Privigen | Cold ethanol fractionation, octanoic | IgG1 67.8% |
| 10% | acid fractionation, depth filtration, | IgG2 28.7% |
| | anion exchange chromatography Cold | IgG3 2.3% |
| | ethanol fractionation, caprylate | IgG4 1.2% |
| | precipitation and depth | |
| | filtration, and anion-exchange | |
| | chromatography and low pH incubation | |
| Gamunex | Cold ethanol fractionation, caprylate | The distribution |
| 10% | precipitation and depth filtration, and | of IgG subclasses |
| | anion-exchange chromatography and | is similar to that |
| | low pH incubation | found in normal |
| | | serum. |
| Octagam | Cold ethanol fractionation | IgG1 60% |
| 10% | | IgG2 32% |
| | | IgG3 7% |
| | | IgG4 1% |

The immunoglobulin preparation prepared by the processes described herein (i.e. without cold ethanol precipitation or chromatographic purification) has an IgG subclass distribution that is substantially the same as that of the pooled plasma, the plasma or normal plasma (IgG1 55%, IgG2 36%, IgG3 5%, IgG4 4%—Miles J & Riches P. Ann Clin Biochem 1994; 31:245-248). It will be understood there may be variations in the IgG subclass profile between batches of pooled plasma. For example, if a proportion of plasma donors have a viral infection or have recently had a viral infection whereby the level of IgG3 may be increased, for example the IgG3 level in the pooled plasma, the plasma or the immunoglobulin preparation may be about 4%, 5%, 6%, 7%, 8%, 9%, or about 10%.

Process does not Involve Chromatography, Ethanol or Caprylate Precipitation

The process described herein is used to prepare pure immunoglobulin preparations from plasma. The process (either when preparing the plasma or in the separation step) does not use ethanol precipitation (e.g. cold ethanol precipitation), chromatographic purification, the use of harsh chemical precipitants such as caprylate, or any combination of these techniques.

In one embodiment the process does not comprise ethanol precipitation, for example cold ethanol precipitation.

In one embodiment the process does not comprise caprylate precipitation, or any step that comprises caprylate or a salt, derivative or analogue thereof.

In one embodiment the process does not comprise chromatography, for example anion exchange chromatography, cation exchange chromatography, size exclusion chromatography, or affinity chromatography (e.g. using protein A, G, A/G and/or L).

### Further Processing

In some embodiments the harvested immunoglobulin solution is further processed. For example, it may be concentrated or subjected to a buffer exchange. Any method of concentrating the harvested immunoglobulin solution may be used, for example ultrafiltration.

In some embodiments it may additionally or alternatively be desirable to perform buffer exchange to, for example, exchange the buffer in harvested immunoglobulin solution for a buffer that is more suitable for use in a therapeutic product or for long term storage of the immunoglobulin solution.

In one embodiment the buffer in the harvested immunoglobulin solution is exchanged for a glycine buffer. The glycine buffer may comprise 3.86 kg glycine and 0.18 kg of 5 M HCl in a total volume of 206 L. A skilled person will be able to alter the buffer volumes.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A process for the production of at least 95% pure immunoglobulin from plasma in a single electroseparation step; the process comprising:
a. applying the plasma to a first fluid stream of an electroseparation system, the electroseparation system comprising:
a separation membrane disposed between a cathode and an anode, the separation membrane having defined pore sizes;
a first restriction membrane disposed between the cathode and the separation membrane and defining a first fluid path for a first fluid stream wherein the first restriction membrane prevents molecules in the first fluid stream contacting the cathode;
a second restriction membrane disposed between the anode and the separation membrane and defining a second fluid path for a second fluid stream wherein the second restriction membrane prevents molecules in the second fluid stream contacting the anode; and
b. applying an electric field to the fluid streams to selectively cause positively charged immunoglobulin molecules to cross the separation membrane toward the cathode and into the second fluid stream; or
a. applying the plasma to the second fluid stream of the electroseparation system, and
b. applying an electric field to the fluid streams to selectively cause negatively charged molecules in the plasma to cross the separation membrane toward the anode and into the first fluid stream.

2. The process of claim 1, further comprising harvesting the at least 95% pure immunoglobulin from the first or second stream.

3. The process of claim 1, further comprising preparing the plasma by reducing the pH of pooled plasma to about 4.5 to 7.5.

4. The process of claim 3, wherein the pH is reduced by the addition of a buffer.

5. The process of claim 4, wherein the buffer comprises MES, Bis-tris, and glycine.

6. The process of claim 5, further comprising removal of precipitate from the plasma.

7. The process of claim 6, wherein removal of precipitate comprises filtration or centrifugation, preferably filtration.

8. The process of claim 1, wherein the first and/or second fluid stream comprises a buffer.

9. The process of claim 8, wherein the buffer comprises MES, Bis-tris, and glycine.

10. The process of claim 1, wherein the defined the pore size of the separation membrane is about from about 200 kDa to about 1500 kDa, preferably about 1000 kDa.

11. The process of claim 1, wherein the at least 95% pure immunoglobulin is free of bacteria, virus or prions when harvested from the first or second stream.

12. The process of claim 1, further comprising concentration of the at least 95% pure immunoglobulin.

13. The process of claim 1, wherein the process does not comprise any one or more of cold ethanol precipitation, caprylate precipitation or chromatography.

14. A preparation comprising at least 95% pure immunoglobulin produced by the process of claim 1.

15. The preparation of claim 14 wherein the IgG subclass distribution is substantially the same as the IgG subclass distribution in the plasma.
